(19) United States
(12) Patent Application Publication
(10) Pub. No.: US 2007/0286835 A1
(43) Pub. Date: Dec. 13, 2007

(54) Skin external preparation having hexagonal gel structure

(76) Inventors: Byeong-Deog Park, Dajeon (KR); Jong-Kyung Youm, Dajeon (KR); Shin-Hee Lee, Dajeon (KR)

Correspondence Address: Thomas M. Galgano, Esq.;GALGANO & ASSOCIATES, PLLC, Suite 204, 20 West Park Avenue, Long Beach, NY, 11561, US

(21) Appl. No.: 11/495,100

(22) Filed: Jul. 28, 2006

(30) Foreign Application Priority Data
Jun. 9, 2006 (KR) 10-2006-0051896

(51) Int. Cl. A61K 31/36; A61K 8/42; A61K 8/36

(52) U.S. Cl. 424/70.22; 424/70.31

(57) ABSTRACT

Disclosed is a skin external preparation containing stratum corneum intercellular lipids, ceramide, and having a hexagonal gel structure. More particularly, the skin external preparation having the hexagonal gel structure contains, as stratum corneum intercellular lipid components, ceramide and cholesterol or its derivative and/or fatty acid, and as an emulsifying agent, sorbitan stearate, sucrose cocoate or a mixture thereof. The hexagonal gel structure of the external preparation is in the same form as a lamellar structure formed by stratum corneum intercellular lipids on the actual skin, and this lamellar structure plays a very important role in skin protection and moisturizing functions, and thus provides very useful effects for skin showing a damaged skin barrier function in various skin diseases or various skin types.

### BACKGROUND OF THE INVENTION

1. Field of the Invention

The present invention relates to a skin external preparation containing stratum corneum intercellular lipid, ceramide, and having a hexagonal gel structure, and more particularly to a skin external preparation having a hexagonal gel structure, which contains, as stratum corneum intercellular lipids, ceramide and cholesterol or its derivative and/or fatty acid, and as an emulsifying agent, sorbitan stearate, sucrose cocoate or a mixture thereof. The hexagonal gel structure according to the present invention is in the same form as a lamella structure formed by stratum corneum intercellular lipids on the actual skin.

2. Description of the Prior Art

The stratum corneum layer is broadly divided into a domain consisting of corneocytes, and a domain consisting of stratum corneum intercellular lipids. This structure of the stratum corneum is often described by the “Brick and Mortar Model”. Generally, the main function of the stratum corneum is divided into a defensive (protective) function to provide a barrier against an external environment, and a moisturizing function to maintain moisture on the skin. The lamella structure formed by corneocyte intercellular lipids plays the most important role in these protective and moisturizing functions (skin barrier functions) of the stratum corneum (Elias P M, Lipid and the epidermal permeability barrier, Arch dermatol Res 1981; 27:95-117). In addition, these stratum corneum intercellular lipids consist of ceramide, cholesterol and its derivatives, fatty acid and the like, and among them, ceramide is known as a very important component forming about 50% of the total lipid.

Generally, the abnormalities of the skin barrier function have a close connection with the abnormality of the stratum corneum intercellular lipids, and if the skin barrier function becomes weakened due to any cause, various skin diseases will occur. Particularly, atopic dermatitis is understood to be mainly caused by damage to the skin barrier, resulting from a reduction in the ceramide content of the stratum corneum (Ogawa H and Yoshiike T, A speculative viex lf atopic dermatitis: barrier dysfunction in pathogenesis. J Dermatol Sci 1993; 5:197-204). Thus, it is known that a supply of stratum corneum intercellular lipids such as ceramide to the skin is effective in normalizing the stratum corneum.

In the 1960s, an X-ray diffraction (XRD) method was first introduced for the analysis of the layered structure of stratum corneum intercellular lipids, and in the 1970s, it was found from the results of freeze fracture electron microscopy that stratum corneum intercellular lipids have lamellar structures between corneocytes (Breathnach A S, Coodman T, Stolinsky C, Cross M. freeze fracture replication of stratum corneum of human cells. H Anat 1973; 114:65-81). Since then, it was reported through wide angle XRD (WXRD) studies that the lamella structures between corneocytes are present in an orthorhombic crystalline phase, a hexagonal gel phase and a liquid lamellar phase, according to the arrangement of head groups and the lateral packing of alkyl groups.

It was found from the results of WXRD analysis for the stratum corneum that, 25° C., typical orthorhombic peak patterns (0.375 nm and 0.416 nm) and a liquid lamellar peak pattern (0.46 nm) appeared, and at 45° C., the peak (orthorhombic) of 0.375 nm and the peak (liquid lamellar) of 0.46 nm disappeared and only one peak of 0.412 nm showed transition to a homologous phase in the state of hexagonal gel (White S H, Mirejovski D, and King G J, Structure of lamellar lipid domains and corneocytes envelopes of murine stratum corneum: An X-ray diffraction study, Biochemistry 1988; 27:3725-3732). From the results of XRD experiments at room temperature, it was recognized that the actual lamellar structure of the stratum corneum in present in a mixed phase of an orthorombic structure, a hexagonal gel phase and a liquid lamellar phase. However, it is known from the result of a study by Noren et al. that stratum corneum lipid has a single phase of hexagonal gel, and the mixed phase is present due to phase transition occurring during a process of preparing a sample for XRD photography. The hexagonal phase is firmer than the liquid lamella phase, so that it helps to normalize the stratum corneum by suppressing the moisture loss while strongly protecting the stratum corneum.

Meanwhile, lamella structures, which are currently industrially used, have a liposome form similar to a biological membrane, are frequently studied as a membrane model system and a medium for the transfer of biological substances (R. Sundler, D. papahadjpovlos, Biochim Biophys Acta, 1981; 648-743), and are widely used in pharmaceutical drugs, cosmetics, foods and the like. Such lamellar liposomes are broadly divided into a multi-lamella vesicle (MLV) having a size of 400-3500 nm and a uni-lamella vesicle (ULV) having a size of 100-1000 nm. Among them, MLV is known to have a lamellar structure, a stratum corneum intercellular structure, leading to an affinity for the skin. This liposome has advantages in that, when prepared into nanosized liposome, it can more effectively deliver a physiologically active ingredient into the skin and can encapsulate all kind of physiologically active ingredients which are oil-soluble or water-soluble. On the other hand, it has disadvantages in that it can encapsulate a limited amount of physiologically active ingredients and has low formulation stability, and the stability of physiologically active oil-soluble ingredients therein is low.

Also, there is an attempt to develop a multi-lamellar emulsion as a novel formulation into a skin external preparation. This multi-lamellar emulsion is mainly based on nonionic surfactant-cetostearylalcohol, but it has problems in that, if it has a lamellar structure having a relatively high fluidity, like the liquid lamellar structure, a swelling phenomenon in the emulsion system will occur to increase the viscosity and hardness of the formulation, or an unstable multi-lamellar structure will tend to disappear. Although this multi-lamellar emulsion is, of course, useful due to the advantage of formulation stability superior to a conventional formulation for external preparations, whether it is useful for phase stability and skin function in connection with a hexagonal gel structure, a skin lamellar structure, is not yet clearly established.

### SUMMARY OF THE INVENTION

Accordingly, it is a first object of the present invention to provide a skin external preparation, which is formulated into the same hexagonal gel structure as a stratum corneum intercellular structure, and thus exhibits excellent skin-protective effects and moisturizing function recovery effects for skin showing an impaired barrier function in various skin types and various skin diseases.

A second object of the present invention is to provide a skin external preparation having excellent formulation stability in addition to said first object.

According to one preferred embodiment of the present invention to smoothly achieve said first and second objects, there is provided a skin external preparation having a hexagonal gel structure, which contains, as stratum corneum intercellular lipid components, ceramide and cholesterol or its derivative and/or fatty acid, and as an emulsifying agent, sorbitan stearate and/or sucrose cocoate. Because the skin external preparation according to the present invention has a signal hexagonal gel structure which is the same as a stratum corneum intercellular lipid structure, it shows an excellent effect of recovering an impaired skin barrier function in various skin types and various skin diseases and, at the same time, shows excellent formulation stability, since it is in the form of a multi-lamellar emulsion.

### BRIEF DESCRIPTION OF THE DRAWINGS

FIG. 1 is a WXRD graph showing the typical single hexagonal gel structure of a skin external preparation prepared in Example 1 of the present invention, in which a typical single hexagonal gel peak of about d=4.12 is shown.

FIG. 2 is a polarizing microscope photograph showing a skin external preparation having a hexagonal gel structure, prepared in Example 1 of the present invention, in which a peculiar Maltese Cross can be observed.

FIG. 3 is a graphic diagram showing the comparison of acute skin barrier recovery function between skin external preparations according to Example 1 of the present invention l and Comparative Example 1.

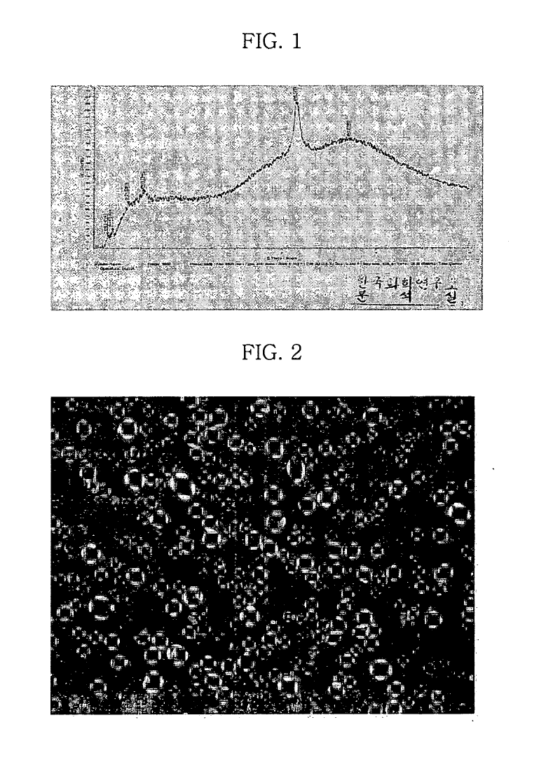

### DETAILED DESCRIPTION OF THE PREFERRED EMBODIMENTS

The inventive skin external preparation having a hexagonal gel structure contains, as stratum corneum intercellular lipids, ceramides represented by each of Formulas 1-5 below, and cholesterol or its derivative and/or fatty acid, and as an emulsifying agent, sorbitan stearate and/or sucrose cocoate, optionally an emulsifying aid such as cetyl alcohol and/or stearyl alcohol. In addition, it may contain general oils, polyhydric alcohol, a thickener and purified water.

The ceramide, which is one of the stratum corneum intercellular lipid components contained in the inventive skin external preparation having a hexagonal gel structure, may be natural ceramide-III represented by Formula 1, or any one of ceramides represented by Formulas 2-5, or any combination thereof.

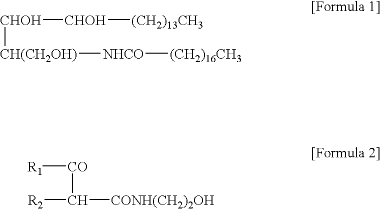

wherein R₁ and R₂ are each individually a straight- or branched-chain alkyl group having 6 to 22 carbon atoms.

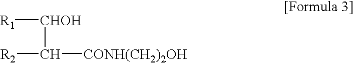

wherein R₁ and R₂ are each individually a straight- or branched-chain alkyl group having 6 to 22 carbon atoms.

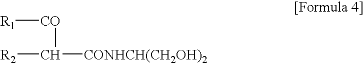

wherein R₁ and R₂ are each individually a straight- or branched-chain alkyl group having 6 to 22 carbon atoms.

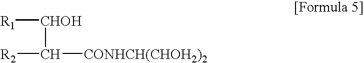

wherein R₁ and R₂ are each individually a straight- or branched-chain alkyl group having 6 to 22 carbon atoms.

Examples of ceramides, which can be used in the present invention, include pseudo-ceramide synthesized to have a structure similar to natural ceramide of the skin, and natural ceramide obtained in nature. Because various kinds of natural ceramide or pseudo-ceramide are currently commercially available, it is to be understood that the present invention is not limited to the above-described ceramides represented by Formulas 1-5, and other ceramides having a structure similar thereto can also be used in the present invention as long as they form a hexagonal gel structure.

Examples of cholesterols or their derivatives which can be used in the present invention include cholesterol, cholesterol sulfate, phytosterol, and other various derivatives having a chemical structure similar to that of cholesterol. In the present invention, preferred are phytosterol and cholesterol, the phytosterol being more preferable in terms of the expression of a hexagonal gel structure.

Also, examples of fatty acid which can be used in the present invention include palmitic acid, cetic acid, stearic acid, and arachidonic acid, which are usable in skin external preparations.

The mixing ratio between the ceramide, the cholesterol or its derivative, and the fatty acid, is not limited to any particular value. As the stratum corneum intercellular lipid components of the skin external preparation according to the present invention, a combination of ceramide and cholesterol or its derivative, or a combination of ceramide or fatty acid is used, and preferably a combination of the above three components (i.e., ceramide, cholesterol or its derivative, and fatty acid) is used.

As the emulsifying agent in the present invention, sorbitan stearate or sucrose cocoate is used alone or in a mixture thereof. In view of efficiency for the formation of a hexagonal gel structure, preferred is sucrose cocoate, and more preferred is a mixture of sorbitan stearate and sucrose cocoate, in which case there is any particular limitation on the mixing ratio between the two components.

The emulsifying aid as an optional component, which can be effectively used in the present invention, contributes to not only the stability of the external preparation, but also the formation of a multi-lamellar structure. As an emulsifying agent, higher fatty alcohol can be generally used, but preferred is cetyl alcohol, stearyl alcohol or cetostearyl alcohol, and more preferred is cetostearyl alcohol.

Thus, the inventive skin external preparation having a hexagonal gel structure contains 0.05-10 wt % of a stratum corneum intercellular lipid component consisting of ceramide and cholesterol or its derivative and/or fatty acid, 0.05-25 wt % of an emulsifying agent consisting of sorbitan stearate and/or sucrose cocoate, and 0.05-20 wt % of at least one emulsifying aid as an optional component selected from the group consisting of cetyl alcohol, stearyl alcohol and cetostearyl alcohol. In the present invention, the stratum corneum intercellular lipid components, the emulsifying agent and the emulsifying agent can be mixed at a weight ratio of 1.0:0.8-3.0:0.01-2.0, and preferably 1.0:1.0-2.0:0.1-2.0, but are not limited thereto.

In addition to said components, to increase utility on the skin and for effective formation and stabilization of a multi-lamellar emulsion, the skin external preparation according to the present invention contains natural oils, including vegetable oil, animal oil and mineral oil, and other synthetic oils. The preferred oil in the present invention is natural vegetable oil.

In the present invention, the weight content of said oils is in the range of 0.5-1.8 times, preferably 0.5-1.8 times, more preferably 1.0-1.6 times, and still more preferably 1.05-1.55 times the total amount of the stratum corneum intercellular lipid component, the emulsifying agent and the emulsifying aid. If the oil content is out of the above range, the external preparation can show an insufficient effect on the skin, or a hexagonal gel structure cannot be expressed.

In addition, the skin external preparation according to the present invention may also contain glycerin, polyhydric alcohol such as butylene glycol, a mixture of various extracts, a thickener, a preservative, an antioxidant, other additives, purified water, and the like.

The inventive skin external preparation having a hexagonal gel structure is prepared in the following manner.

First, the stratum corneum intercellular lipid component consisting of ceramide and cholesterol or its derivative and/or fatty acid, the emulsifying agent consisting of sorbitan stearate and/or sucrose cocoate, and the emulsifying aid consisting of cetyl alcohol, stearyl alcohol or cetostearyl alcohol, are uniformly mixed with each other while dissolving them by heating at 70-100° C., and preferably 85-95° C. Then, oil and/or polyhydric alcohol is added thereto, and the mixture solution is subjected to phase transition emulsification while adding warmed water thereto, or is emulsified while adding the mixed emulsion phase to warmed water. Then, after adding other additives, the mixture is cooled to room temperature, yielding the inventive skin external preparation. The additives, which are mixed with the emulsion phase, can be preferably added by introducing them in the process of adding oil and/or polyhydric alcohol.

Hereinafter, the present invention will be described in further detail with reference to Examples and Comparative Examples. It is to be understood, however, that these examples are for illustrative purposes only, and are not to be construed to limit the scope of the present invention.

### EXAMPLES 1 TO 9 AND COMPARATIVE EXAMPLES 1 TO 3

A stratum corneum intercellular lipid component, an emulsifying agent, an emulsifying aid, general oils, polyhydric alcohol, a thickener and purified water were used at composition ratios shown in Table 1 below so as to prepare the inventive skin external preparations having a hexagonal gel structure.

For this purpose, a stratum corneum intercellular lipid component consisting of ceramide and cholesterol or its derivative and/or fatty acid, an emulsifying agent selected from among sorbitane stearate, stearyl alcohol and a mixture thereof, and an emulsifying aid consisting of cetyl alcohol, stearyl alcohol or cetostearyl alcohol, were weighed and then dissolved by heating them at 85-95° C. Then, polyhydric alcohol was added thereto and uniformly mixed. Then, the mixture solution was emulsified by strong stirring and cooled below 50° C., and a previously hydrated thickener was added thereto and uniformly mixed, yielding skin external preparations. As the ceramid, each of the following three ceramides, which are currently commercially available among the examples represented by said formulas, were used: ceramide (1) represented by Formula 1 (Cermide-IIIB®); ceramide (2) (PC-9S®) represented by Formula 2 wherein R₁=a mixture of —C₁₅H₃₁ and —C₁₇H₃₅, and R₂=a mixture of —C₁₄H₂₉ and —C₁₆H₃₃; ceramide (3) (PC-5SP®) represented by Formula 4 wherein R₁=a mixture of —C₁₅H₃₁ and —C₁₇H₃₅, and R₂=a mixture of —C₁₄H₂₉ and —C₁₆H₃₃. As the thickener, a carboxyvinyl polymer was used.

In Comparative Examples 1-3, the following skin external preparations were prepared, respectively: an external preparation containing no stratum corneum intercellular lipid; an external preparation containing no emulsifying agent; and an external preparation in which the content of oils was 1.5 times as large as the total content of the stratum corneum intercellular lipid, the emulsifying agent and the emulsifying aid.

TABLE 1

| | Exam- | Exam- | Exam- | Exam- | Exam- | | | | | Comp. | Comp. | Comp. |
|---|---|---|---|---|---|---|---|---|---|---|---|---|
| Raw materials | ple 1 | ple 2 | ple 3 | ple 4 | ple 5 | Example 6 | Example 7 | Example 8 | Example 9 | Example 1 | Example 2 | Example 3 |
| Ceramide (1) | 0.5 | 0.5 | 0.5 | — | — | 0.8 | 0.4 | — | 0.6 | — | 0.5 | 0.5 |
| Ceramide (2) | — | — | — | 0.5 | — | — | — | — | — | — | — | — |
| Ceramide (3) | — | — | — | — | 0.5 | — | — | 0.6 | — | — | — | — |
| Phytosterol | 0.2 | — | 0.2 | 0.2 | 0.2 | — | 0.2 | 0.1 | 0.4 | — | 0.2 | 0.2 |
| Cholesterol | — | — | — | — | — | 0.4 | — | — | — | — | — | — |
| Stearic acid | 1.6 | 1.6 | 1.6 | 1.6 | 1.6 | 0.8 | 1.4 | 1.5 | 1.3 | — | 1.6 | 1.6 |
| Sorbitan stearate | 2.25 | 2.0 | 2.25 | 2.25 | 2.25 | — | 2.5 | 1.5 | 4.0 | 2.25 | — | 2.0 |
| Sucrose cocoate | 0.25 | 0.5 | 0.25 | 0.25 | 0.25 | 2.5 | — | 1.5 | 1.0 | 0.25 | — | 0.5 |
| Cetyl alcohol | 1.5 | 2.8 | — | 1.5 | 1.5 | 2.5 | — | 1.5 | 0.5 | 1.5 | 1.5 | 2.8 |
| Stearyl alcohol | 0.5 | 0.7 | 0.3 | 0.5 | 0.5 | — | 2.5 | 0.5 | 1.5 | 0.5 | 0.5 | 0.7 |
| Meadowfoam | 3.0 | 2.0 | 2.5 | 3.0 | 3.0 | 4.0 | 4.0 | 1.5 | 4.0 | 3.0 | 2.0 | 7.0 |
| seed oil | | | | | | | | | | | | |
| Grape seed oil | 3.0 | 2.0 | 2.5 | 3.0 | 3.0 | 4.0 | 4.0 | 1.5 | 4.0 | 3.0 | 2.0 | 8.0 |
| Squalane | 1.5 | 1.0 | 1.2 | 1.5 | 1.5 | 2.5 | 2.5 | 0.7 | 3.5 | 1.5 | 1.0 | 3.0 |
| Glycerin | 2.5 | 2.5 | 2.5 | 2.5 | 2.5 | 2.5 | 2.5 | 2.5 | 2.5 | 2.5 | 2.5 | 2.5 |
| 1,3-butylene | 2.5 | 2.5 | 2.5 | 2.5 | 2.5 | 2.5 | 2.5 | 2.5 | 2.5 | 2.5 | 2.5 | 2.5 |
| glycol | | | | | | | | | | | | |
| Thickener | 0.2 | 0.2 | 0.2 | 0.2 | 0.2 | 0.2 | 0.2 | 0.2 | 0.2 | 0.2 | 0.2 | 0.2 |
| Purified water | Balance | Balance | Balance | Balance | Balance | Balance | Balance | Balance | Balance | Balance | Balance | Balance |

### Test Example 1

### Examination of Hexagonal Gel Structure

To examine whether the emulsions prepared in Examples 1-9 and Comparative Examples 1-3 had a hexagonal gel structure formed therein, WXRD was carried out.

For this purpose, each of the emulsions prepared in Examples 1-9 and Comparative Examples 1-3 was placed in a capillary (Mark-Rohrchem aus Glas no 10, 80 mm length, 0.7 mm auBen, Article no. 4007807, Hilgenberg, Germany), and the sample temperature was controlled with a D8 discover XRD for WXRD at high temperature conditions. Cu-Kα radiation was used at 40 kV and 40 mA. A collimater was set at 0.3 mm, and measurement was carried out at 150 sec/scan. Wavelengths emitted from the samples were measured while transmitting them through a 30-cm helium gas tube.

Meanwhile, whether a liquid crystal as a multi-lamellar emulsion was formed was observed under a polarizing microscope. Generally, when a multi-lamellar emulsion is formed, a peculiar Maltese cross can be observed in a polarizing microscope. To observe the Maltese cross, a polarizing microscope (Optiphto-2, Nikon) was used, and a suitable amount of each of the prepared emulsions was placed on a slide glass, covered with a cover glass, lightly pressed and then observed at 500× magnification.

As a result, as shown in Table 2 below, the WXRD results and the presence or absence of the multi-lamellar structure could be confirmed.

TABLE 2

| | Exam- | Exam- | Exam- | Exam- | Exam- | | | | | Comp. | Comp. | Comp. |
|---|---|---|---|---|---|---|---|---|---|---|---|---|
| Items | ple 1 | ple 2 | ple 3 | ple 4 | ple 5 | Example 6 | Example 7 | Example 8 | Example 9 | Example 1 | Example 2 | Example 3 |
| Single hexagonal | ⊚ | ◯ | ⊚ | ⊚ | ⊚ | ⊚ | ⊚ | ◯ | ⊚ | ◯ | X | X |
| gel peak | | | | | | | | | | | | |
| Multi-lamellar | ⊚ | ◯ | ◯ | ⊚ | ⊚ | ⊚ | ⊚ | ◯ | ⊚ | ◯ | X | X |
| liquid crystal | | | | | | | | | | | | |
| structure | | | | | | | | | | | | |

* good formation of structure: ⊚, formation of structure: ◯, not formation of structure: X

### Test Example

### Test of Skin Barrier Recovery

To evaluate the skin utility of the inventive skin external preparations having a hexagonal gel structure, the test of acute skin barrier recovery was carried out. In the test, nude mice were used as an animal model, and a tape stripping method was applied, in which the skin barrier was damaged by removing the stratum corneum from the back of the nude mice using a cellophane tape. In this method, when the stratum corneum is removed by tape stripping, transepidermal water loss (TWEL) is rapidly increased, and thus the recovery of battier function can be comparatively evaluated by applying the external product on the damaged skin. Example 1 and Comparative Example 1 were comparatively evaluated. Comparative Example 1 is a multi-lamellar emulsion based on nonionic surfactant-cetosterylalcohol and it was found that Comparative Example 1 is a preparation having a hexagonal gel structure formed therein. By performing a comparative test with Comparative Example 3, the importance of stratum corneum intercellular lipid components can be evaluated.

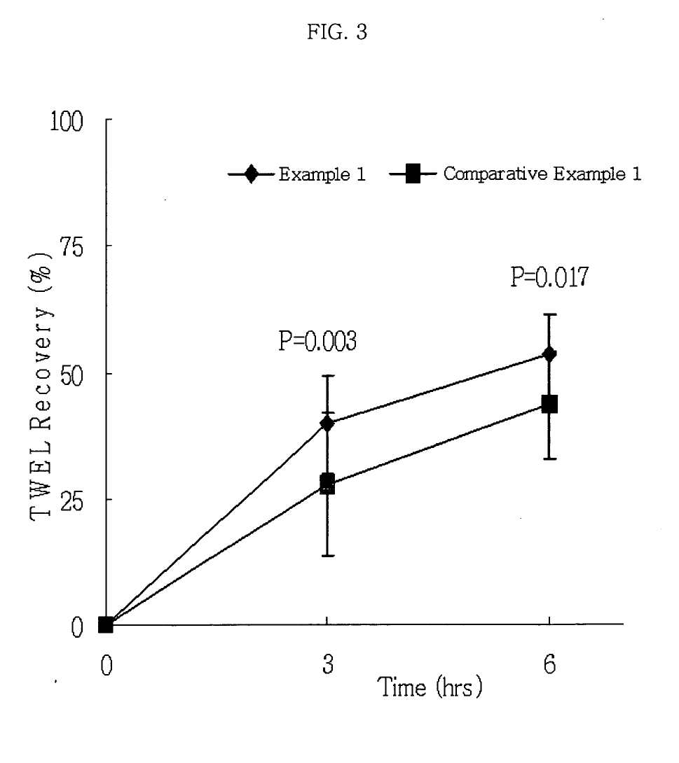

The test results are shown in FIG. 3. As shown in FIG. 3, when Example 1 and Comparative Example 3 were applied just after the skin barrier impairment and compared with each other with respect to barrier recovery function, Example 1 showed very useful results which were significantly different from of Comparative Example 3.

### Test Example 3

### Stability Test of Preparations

The formulation stability of the inventive emulsions having a lamellar structure was measured in the following manner. The following samples were measured for phase stability and liquid crystal stability: samples obtained by placing Examples 1-9 and Comparative Examples 1-3 in opaque glass containers and storing them in incubators at 25° C. and 45° C. for 24 hours; and samples obtained by placing Examples 1-9 and Comparative Examples 1-3 in a chamber having a temperature cycle of −20° C. for 24 hr and 25° C. for 24 hr and repeating the temperature cycle 20 times.

As a result, Examples were all stable or very stable, but Comparative Examples 1-3 all showed phase separation, and the liquid crystal structure of Comparative Example 1 gradually disappeared with the passage of time.

TABLE 3

| | | Exam- | Exam- | Exam- | Exam- | Exam- | | | | | Comp. | Comp. | Comp. |
|---|---|---|---|---|---|---|---|---|---|---|---|---|---|
| Items | | ple 1 | ple 2 | ple 3 | ple 4 | ple 5 | Example 6 | Example 7 | Example 8 | Example 9 | Example 1 | Example 2 | Example 3 |
| Phase | 25° C. | ⊚ | ◯ | ◯ | ⊚ | ◯ | ⊚ | ⊚ | ◯ | ⊚ | ◯ | X | ◯ |
| stability | 45° C. | ⊚ | ◯ | ◯ | ⊚ | ◯ | ⊚ | ⊚ | ◯ | ◯ | X | X | X |
| | −20 to | ⊚ | ◯ | ◯ | ⊚ | ◯ | ⊚ | ⊚ | ◯ | ⊚ | X | X | X |
| | 25° C. | | | | | | | | | | | | |
| Liquid | 25° C. | ⊚ | ◯ | ◯ | ⊚ | ◯ | ⊚ | ⊚ | ◯ | ⊚ | ◯ | X | ◯ |
| crystal | 45° C. | ⊚ | ◯ | ◯ | ⊚ | ◯ | ⊚ | ⊚ | ◯ | ◯ | X | X | X |
| stability | −20 to | ⊚ | ◯ | ◯ | ⊚ | ◯ | ⊚ | ⊚ | ◯ | ⊚ | X | X | X |
| | 25° C. | | | | | | | | | | | | |

* very stable: ⊚, stable: ◯, unstable: X

### Test Example 3

### Skin Irritation Test

To measure the skin irritability of the inventive skin preparations having a hexagonal gel structure, 30 healthy adult men and women had the upper arm onto which Haye's chambers stained with a predetermined amount (about 2.0 g), which is used for the patch test, of the individual preparations of Examples 1-9 and Comparative Examples 1-3 were applied for 48 hours. After an elapse of 30 minutes to one hour since the Haye's chambers were removed, the change of the complexion was observed with unaided eyes for the primary finding, and again after more 48 hours for the secondary finding.

As shown in Table 4 below, the preparations of Examples 1-9 and Comparative Examples 1-3 all showed no skin irritability.

TABLE 4

| | | Evaluation results | | | | | | | | | | |
|---|---|---|---|---|---|---|---|---|---|---|---|---|
| | | | | | | Slightly | | | | | | |
| | Number | No irritation | | Uncertain | | irritable | | Irritable | | Severely irritable | | |
| | of test | | Second- | | Second- | | Second- | | Second- | | Second- | Degree of |
| Samples | subjects | Primary | ary | Primary | ary | Primary | ary | Primary | ary | Primary | ary | irritation |
| Example 1 | 30 | 30 | 30 | | | | | | | | | No irritation |
| Example 2 | 30 | 30 | 30 | | | | | | | | | No irritation |
| Example 3 | 30 | 30 | 30 | | | | | | | | | No irritation |
| Example 4 | 30 | 30 | 30 | | | | | | | | | No irritation |
| Example 5 | 30 | 29 | 30 | 1 | | | | | | | | No irritation |
| Example 6 | 30 | 30 | 30 | | | | | | | | | No irritation |
| Example 7 | 30 | 30 | 30 | | | | | | | | | No irritation |
| Example 8 | 30 | 30 | 30 | | | | | | | | | No irritation |
| Example 9 | 30 | 30 | 30 | | | | | | | | | No irritation |
| Comp. | 30 | 30 | 30 | | | | | | | | | No irritation |
| Example 1 | | | | | | | | | | | | |
| Comp. | 30 | 30 | 30 | | | | | | | | | No irritation |
| Example 2 | | | | | | | | | | | | |
| Comp. | 30 | 30 | 30 | | | | | | | | | No irritation |
| Example 3 | | | | | | | | | | | | |

As described above, the present invention provides the skin external preparation having a hexagonal gel structure, which contains the stratum corneum intercellular lipid component consisting of ceramide and cholesterol or its derivative and/or fatty acid, and the emulsifying agent selected from among sorbitan stearate, sucrose and a mixture thereof. It was found that the inventive skin external preparation has a clear single hexagonal gel structure, provides skin barrier recovery, shows very excellent formulation stability and has no skin irritability. Thus, the skin external preparation according to the present invention has a hexagonal gel structure equal to a stratum corneum intercellular lipid structure, and thus provides a useful external preparation for skin protection and moisturizing function recovery to skin showing a damaged skin barrier function in various skin types or various skin diseases. In addition, it has excellent formulation stability, because it is made of a preparation in the form of a multi-lamellar emulsion.

Although a preferred embodiment of the present invention has been described for illustrative purposes, those skilled in the art will appreciate that various modifications, additions and substitutions are possible, without departing from the scope and spirit of the invention as disclosed in the accompanying claims.

1. A skin external preparation having a hexagonal gel structure, which contains, as a stratum corneum intercellular lipid component, ceramide, and as an emulsifying agent, sorbitan stearate and/or sucrose cocoate.

2. The skin external preparation of claim 1, which further contains, as said stratum corneum intercellular lipid component, cholesterol or its derivative and/or fatty acid.

3. The skin external preparation of claim 1, wherein said ceramide is a natural ceramide-III represented by Formula 1, or at least one selected from the group consisting of pseudo-ceramides represented by Formulas 2 to 5: 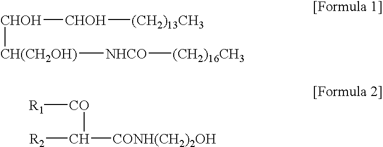
wherein R₁ and R₂ are each individually a straight- or branched alkyl group having 6 to 22 carbon atoms; 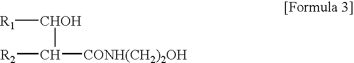
wherein R₁ and R₂ are each individually a straight- or branched alkyl group having 6 to 22 carbon atoms; 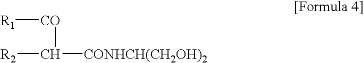
wherein R₁ and R₂ are each individually a straight- or branched alkyl group having 6 to 22 carbon atoms; 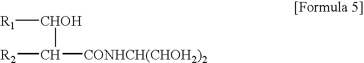
wherein R₁ and R₂ are each individually a straight- or branched alkyl group having 6 to 22 carbon atoms.

4. The skin external preparation of claim 2, wherein said cholesterol derivative is cholesterol sulfate and/or phytosterol.

5. The skin external preparation of claim 2, wherein said fatty acid is selected from the group consisting of palmitic acid, cetic acid, stearic acid and arachidonic acid.

6. The skin external preparation of claim 1, which further comprises, as an emulsifying aid, a member selected from the group consisting of cetyl alcohol, stearyl alcohol and cetostearyl alcohol.

7. The skin external preparation of claim 6, which contains 0.05-10 wt % of the stratum corneum intercellular lipid component, 0.05-25 wt % of the emulsifying agent, and 0.05-20 wt % of the emulsifying aid.

8. The skin external preparation of claim 6, wherein the stratum corneum intercellular lipid component, the emulsifying agent and the emulsifying aid is mixed with each other at a mixing ratio of 1.0:0.8-3.0:0.01-2.0.

9. The skin external preparation of claim 1, which further comprises oil.

10. The skin external preparation of claim 6, which further comprises oil, the weight content of which is in a range of 0.5-1.8 times the total amount of the stratum corneum intercellular lipid component, the emulsifying agent and the emulsifying aid.

11. The skin external preparation of claim 10, wherein the weight content of said oil is in a range of 1.0-1.6 times the total amount of the stratum corneum intercellular lipid component, the emulsifying agent and the emulsifying aid.

12. The skin external preparation of claim 1, which is in the form of a multi-lamellar emulsion.

13. The skin external preparation to claim 2, which is in the form of a multi-lamellar emulsion.

14. The skin external preparation of claim 2, wherein said ceramide is a natural ceramide-III represented by Formula 1, or at least one selected from the group consisting of pseudo-ceramides represented by Formulas 2 to 5: 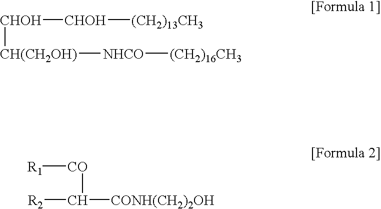
wherein R₁ and R₂ are each individually a straight- or branched alkyl group having 6 to 22 carbon atoms; 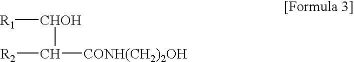
wherein R₁ and R₂ are each individually a straight- or branched alkyl group having 6 to 22 carbon atoms; 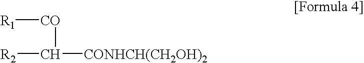
wherein R₁ and R₂ are each individually a straight- or branched alkyl group having 6 to 22 carbon atoms; 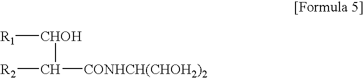
wherein R₁ and R₂ are each individually a straight- or branched alkyl group having 6 to 22 carbon atoms.

15. The skin external preparation of claim 2, which further comprises, as an emulsifying aid, a member selected from the group consisting of cetyl alcohol, stearyl alcohol and cetostearyl alcohol.

16. The skin external preparation of claim 2, which further comprises oil.
